(19) United States
(12) Patent Application Publication
(10) Pub. No.: US 2022/0289486 A1
(43) Pub. Date: Sep. 15, 2022

(54) SEQUESTRATION OF DE-OILED ALGAE BODIES

(71) Applicant: ExxonMobil Research and Engineering Company, Annandale, NJ (US)

(72) Inventor: Patrick L. Hanks, Bridgewater, NJ (US)

(21) Appl. No.: 17/199,628

(22) Filed: Mar. 12, 2021

(51) Int. Cl. B65G 5/00; C12N 1/12; C12P 7/64; E21B 41/00

(52) U.S. Cl. CPC B65G 5/00; C12N 1/12; C12P 7/64; E21B 41/0057

(57) ABSTRACT

Disclosed herein are processes and systems for disposal of de-oiled algae bodies used for production of lipids and other bioproducts. An exemplary process may include providing a de-oiled algae body stream comprising de-oiled algae bodies, processing the de-oiled algae bodies by de-watering the de-oiled algae bodies, slurrying the de-oiled algae bodies with water to produce an algae body disposal stream, and then pumping the de-oiled algae bodies into a salt cavern.

### FIELD OF THE INVENTION

This application relates to processes and systems for disposal of de-oiled algae bodies used for production of lipids and other bioproducts.

### BACKGROUND OF THE INVENTION

Algae have been developed as efficient micro factories for production of biological products. Algae utilize carbon dioxide, water, and sunlight as input to photosynthesize fuel for the algae metabolic activities. In turn, the algae are able to metabolize various biological products necessary for life, including proteins and fats. Some algae have been utilized in the production of lipids for development of alternative pathways for sustainable fuel production. The lipids produced within the algae cells may be separated from the algae bodies, which may include everything that is not the desired lipids. Worldwide interest in alternative biofuels has led to a large increase in algae biomass requiring disposal. While there may be some secondary uses for the de-oiled algae bodies, presently used methods do not sequester the carbon content of the algae biomass.

### SUMMARY OF THE INVENTION

Disclosed herein are example methods and systems for disposal of de-oiled algae bodies used for production of lipids and other bioproducts. A method may include providing a de-oiled algae body stream comprising de-oiled algae bodies; and pumping the de-oiled algae bodies into a salt cavern.

Further disclosed herein is an example method which may include providing a de-oiled algae body stream comprising de-oiled algae bodies; and pumping the de-oiled algae bodies into an underground storage location.

A method comprising: providing a de-oiled algae body stream comprising de-oiled algae bodies; processing the de-oiled algae bodies by slurrying the de-oiled algae bodies with water to produce an algae body disposal stream; and pumping the de-oiled algae bodies into a salt cavern.

### BRIEF DESCRIPTION OF THE DRAWINGS

The drawings illustrate certain aspects of the present disclosure and should not be used to limit or define the disclosure.

FIG. 1 is a block flow diagram of a process for disposal of de-oiled algae bodies used for production of lipids and other bioproducts.

FIG. 2 is schematic illustration of a system for disposal of de-oiled algae bodies in a salt cavern.

### DETAILED DESCRIPTION OF THE INVENTION

The following is a detailed description of the disclosure provided to aid those skilled in the art in practicing the present disclosure. Those of ordinary skill in the art may make modifications and variations in the embodiments described herein without departing from the spirit or scope of the present disclosure. Unless otherwise defined, all technical and scientific terms used herein have the same meaning as commonly understood by one of ordinary skill in the art to which this disclosure belongs. The terminology used in the description of the disclosure herein is for describing particular embodiments only and is not intended to be limiting of the disclosure.

This application relates to methods and systems for disposal of de-oiled algae bodies used for production of lipids and other bioproducts. As discussed above, algae are utilized for production of biofuels may also produce a large volume of algae biomass waste, such as de-oiled algae bodies, that must be disposed of. There are several solutions currently used to dispose of the de-oiled algae bodies, however, none of the presently used method sequester the carbon present in the de-oiled algae bodies.

In some examples, the de-oiled algae bodies may be utilized as fertilizer for crops. The nitrogen and phosphorus content in the de-oiled algae bodies is already fixed, thereby providing useable nutrients. However, there are usually significant amounts of salts associate with the de-oiled algae, especially when the de-oiled algae bodies are from marine algae, which may limit the usefulness of the de-oiled algae bodies as a fertilizer. Additionally, the carbon component of the de-oiled algae bodies decomposes to carbon dioxide and is therefore not sequestered. De-oiled algae bodies may also be used for soil amendment which is a similar application to fertilizer where the carbon component decomposes to carbon dioxide. Another use for de-oiled algae bodies may be in animal feed. While the de-oiled algae bodies have high dollar value as feed, the carbon component is still digested to carbon dioxide by respiration in the animal.

There may be uses for de-oiled algae bodies as fillers for composite materials such as cement, however, the de-oiled algae bodies usually exhibit poor filler properties compared to other filler materials available. There may be some applications of using the de-oiled algae bodies in biodegradable materials, such as shoes, but the commodity of scale is not large enough when compared to the volume of de-oiled algae body produced for biofuels.

De-oiled algae bodies themselves may be utilized for fuel in several ways. For example, the de-oiled algae bodies may undergo anerobic digestion. However, the high salt and nitrogen content of the algae de-oiled bodies typically requires dilution with equal parts cellulosic material, and the resulting biogas when burned does not sequester carbon. Directly burning de-oiled algae bodies for heat, power, or gasification directly releases carbon dioxide to the atmosphere. Further, the relatively high nitrogen content of the algae may lead to high NOx emissions requiring additional emission control technologies to reduce atmospheric smog. De-oiled algae bodies may be pyrolyzed to produce a bio-char which can be considered sequestered carbon. However, the resulting liquids are rich in heteroatoms and oxygen and have low heating values which are difficult to use without extensive hydrogenation. Hydrogenation is energy intensive and thus carbon dioxide intensive. Further, the pyrolysis itself is a high energy input which typically results in substantial emissions.

At present, none of the practiced techniques for algae body disposal sequester carbon to ensure that the carbon content of the algae bodies does not end up back in the atmosphere as carbon dioxide. The present disclosure utilizes techniques which may increase the carbon sequesterability of de-oiled algae bodies with reduced carbon emissions versus techniques presently used for disposal of de-oiled algae bodies.

The methods described herein may mitigate all of the above mentioned problems with current disposal techniques for de-oiled algae bodies, including near total sequestration of the carbon from the de-oiled algae bodies. One method described herein is to introduce de-oiled algae bodies into an underground salt cavern which is nearly impermeable to outside fluids. The salt cavern allows long term, permanent storage for the algae bodies, thereby totally sequestering the carbon associated with the de-oiled algae bodies.

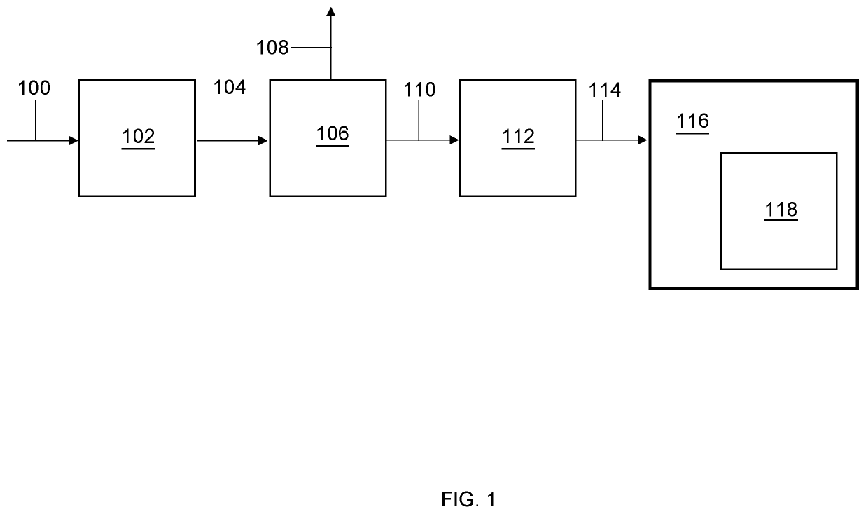

FIG. 1 illustrates an example process whereby algae is grown to produce biological products, such as lipids, with disposal of the resultant de-oiled algae bodies. Algae growth medium stream 100 containing carbon dioxide, water, nutrients, and other compounds required for algae growth may be introduced into algae bioreactor 102. In algae bioreactor 102, algae may be allowed to grow to produce the desired biological products. From algae bioreactor 102, algae stream 104 containing the algae grown in algae bioreactor 102 may be transported to extraction unit 106. In extraction unit 106, the algae may be exposed to heat, mechanical agitation, chemical treatment, or any other suitable technique for separating the biological products from the algae bodies to thereby produce de-oiled algae bodies. A result of the de-oiling process in extraction unit 106 may be that at least a portion of the oil from the biological products, which may include oils such as lipids and triglycerides, are removed from the algae, leaving behind algae bodies. The particular amount of oil removed from the algae may be a function of the extraction technique utilized. Extraction unit 106 may contain several pieces of equipment configured to extract at least a portion of the biological products from algae. Example equipment may include an expresser configured to apply pressure to the algae to break open the algae to liberate biological products from the algae. Expresser equipment may include screw-type, expeller, and piston configurations, for example. Other equipment may include ultrasonication equipment. A method for producing biological products from algae may be ultrasonication in the presence of a solvent whereby the algae may be exposed to ultrasonic waves creating cavitation on the surface of the algae thereby breaking open the algae to liberate biological products. The biological products may thereafter be absorbed into the solvent. The solvent may be any suitable solvent which is capable of absorbing at least a portion of the biological products, including organic solvents such as alkanes and aromatics, for example. Other equipment in extraction unit 106 may include liquid-liquid solvent extraction equipment such as mixer-settler units, columns, and centrifugal extractors. Liquid-liquid solvent extraction methods may employ a solvent which is able to absorb at least a portion of the biological products from the algae such as those solvents described above. Biological product stream 108 may be withdrawn from extraction unit 106 and de-oiled algae body stream 110 may be sent to disposal unit 112. Biological product stream 108 may include a lipid and/or triglyceride, for example. In disposal unit 112, the de-oiled algae bodies from de-oiled algae body stream 110 may be slurried with water to enable pumping and pumped to disposal well 116. Disposal unit 112 may process the de-oiled algae bodies to make the de-oiled algae bodies suitable for disposal in disposal well 116. Some processes employed in disposal unit 112 may include shearing, de-watering, watering, and addition of chemical additives, for example. Disposal unit 112 may include various equipment such as pumps, vessels, masticators, settling units, sedimentation units, chemical additive pumps, dehydrators, and other equipment associated with shearing, de-watering, watering, and addition of chemical additives.

Shearing may reduce the size of the de-oiled algae bodies to enable pumping, for example. In some embodiments the de-oiled algae bodies may be sheared to reduce the size of the de-oiled algae bodies to an average size of less than 100 microns, where size herein refers to an average diameter. Alternatively, the de-oiled algae bodies may be sheared to less than 75 microns, less than 50 microns, less than 25 microns, less than 15 microns, or less than about 1 micron. Another process may be to de-water the de-oiled algae bodies to remove a fraction of water present in the de-oiled algae bodies. De-watering may remove at least a portion of the water associated with de-oiled algae body stream 110, thereby reducing the water content of de-oiled algae body stream. Alternatively, the de-oiled algae bodies may have water added to rehydrate the de-oiled algae bodies. It may be advantageous to reduce the volume of disposal of the de-oiled algae bodies such that the volume of the de-oiled algae bodies in the disposal well may be maximized. In general, the water content may be adjusted to where the algae bodies remain pumpable but the total volume reduced. The water content of the de-oiled algae bodies may be adjusted to less than 50% by weight water. Alternatively, the de-oiled algae bodies may be reduced to less than 40% by weight water, less than 30% by weight water, or less than 25% by weight water, for example. Another process employed in disposal unit 112 may be addition of flocculating agents. Some suitable flocculating agents may include, but are not limited to, alum, aluminum chlorohydrate, aluminum sulphate, calcium oxide, calcium hydroxide, iron chloride, iron sulfate, polyacrylamide, sodium aluminate, and sodium silicate, for example. Flocculating agents may be used to break emulsion layers or agglomerates in disposal stream 114 to promote settling when the de-oiled algae bodies are eventually disposed in the salt cavern. The flocculating agents may be added in amounts suitable to promote settling, such as from about 0.1 PPM to about 100 PPM. Alternatively, from about 0.1 PPM to about 1 PPM, about 1 PPM to about 50 PPM, or about 50 PPM to about 100 PPM, for example. From disposal unit 112, disposal stream 114 containing de-oiled algae bodies may be withdrawn and pumped into disposal well 116.

Oil and natural gas that is obtained from oil wells may be stored in an underground oil and natural gas storage facility. There are three general types of underground oil and natural gas storage facilities, including aquifers, depleted oil or gas field reservoirs, and caverns formed in salt or carbonate formations. These underground facilities are characterized primarily by their capacity or the amount of oil or natural gas that may be held in the facility, and their deliverability or the rate at which the oil or natural gas, or any other fluids pumped within the facility may be deposited.

Salt caverns are typically created by drilling a well into a salt formation, e.g., a salt dome or salt bed, and using water to dissolve and extract salt from the salt formation, leaving a large empty space, or cavern, behind. While salt caverns tend to be costly compared to aquifers and reservoirs, they also have very high deliverability, withdrawal rates, and injection rates. In addition, the walls of a salt cavern have a high degree of strength and resilience to degradation and are essentially impermeable, allowing little material pumped into the salt formation to escape from the cavern unless purposefully extracted. Salt cavern storage facilities are usually only about one hundredth of the size of aquifer and reservoir storage facilities, averaging about three hundred to six hundred feet in diameter and two thousand to three thousand feet in height. Accordingly, the capacity of salt caverns may range between around one million barrels to twenty million barrels of material.

Disposal well 116 may include a salt cavern as described above. There may be several advantages to disposing of the de-oiled algae bodies in a salt cavern, including nearly complete sequestration of the carbon content of the de-oiled algae bodies. The de-oiled algae bodies from disposal unit 112 may be directly pumped into a salt cavern without mechanical agitation or pump around from or within the salt cavern to maximize sedimentation. The de-oiled algae bodies may be allowed to settle and separate from the bulk water phase thereby forming a layer of de-oiled algae bodies in the bottom of the salt cavern. As additional de-oiled algae bodies slurried with water are pumped into the salt cavern, the additional de-oiled algae bodies may also separate and form additional layers of sediment within the salt cavern. The water which separates from the de-oiled algae bodies from sedimentation may be withdrawn from the salt cavern over time, thereby reducing the total volume of material in the salt cavern and maximizing the volume of de-oiled algae bodies able to be stored.

Over the course of several hundred years, the de-oiled algae bodies may break down, either from geothermal effects or by bacterial growth, to form simple hydrocarbons such as methane, ethane, and propane, for example, which may then be extracted.

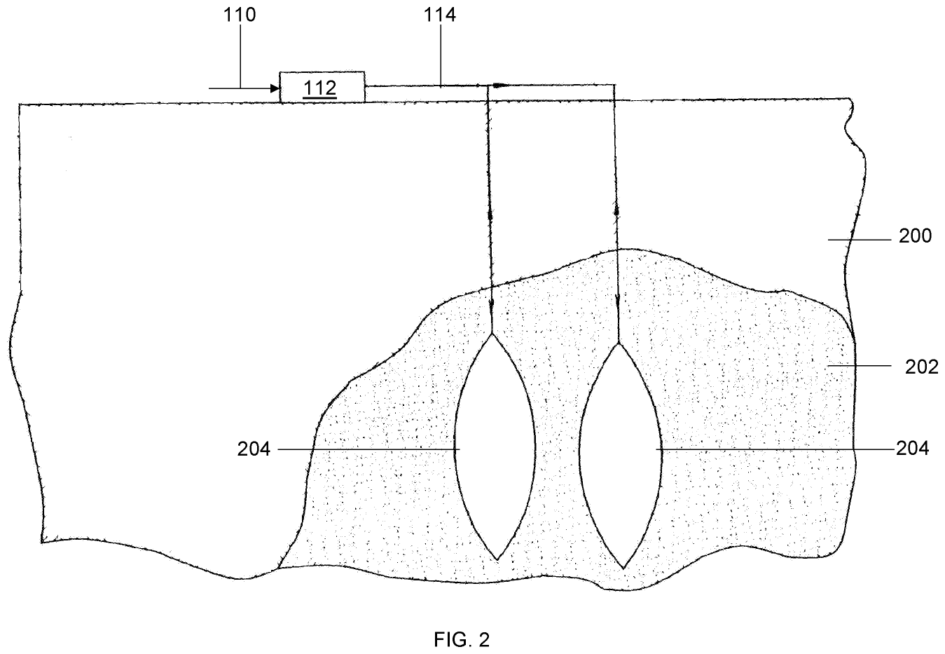

FIG. 2 is a schematic illustration of a system for disposal of de-oiled algae bodies in a salt cavern. As illustrated in FIG. 2, subterranean formation 200 may include a salt dome 202. Salt dome 202 may include salt caverns as discussed above. De-oiled algae stream 110 may be introduced into disposal unit 112. From disposal unit 112, disposal stream 114 may be introduced into salt cavern 204. Disposal unit 112 may include the requisite pumping equipment required to introduce disposal stream 114 into salt cavern 204. While illustrated as a salt cavern in FIG. 2, disposal stream 114 may be introduced into any suitable underground storage location including an aquifer, a depleted oil or gas field reservoir, or a cavern formed in a carbonate formation or salt cavern.

Accordingly, the preceding description describes methods and systems for disposal of de-oiled algae bodies used for production of lipids and other bioproducts. The processes and systems disclosed herein may include any of the various features disclosed herein, including one or more of the following embodiments.

Embodiment 1. A method comprising: providing a de-oiled algae body stream comprising de-oiled algae bodies; and pumping the de-oiled algae bodies into a salt cavern.

Embodiment 2. The method of embodiment 1, further comprising processing the de-oiled algae bodies by de-watering the de-oiled algae bodies prior to pumping the de-oiled algae bodies.

Embodiment 3. The method of any of embodiments 1-2, further comprising slurrying the de-oiled algae bodies with water prior to pumping the de-oiled algae bodies.

Embodiment 4. The method of embodiment 3, wherein the de-oiled algae bodies are slurried with water to between about 25% to about 50% by weight water.

Embodiment 5. The method of any of embodiments 1-3, further comprising reducing the de-oiled algae bodies in size prior to pumping the de-oiled algae bodies.

Embodiment 6. The method of embodiment 5, wherein the de-oiled algae bodies are reduced in size to an average of less than about 15 microns prior to pumping the de-oiled algae bodies.

Embodiment 7. The method of any of embodiments 1-6, further comprising adding a flocculating agent to the de-oiled algae bodies prior to pumping the de-oiled algae bodies.

Embodiment 8. The method of embodiment 7, wherein the flocculating agent is selected from the group consisting of alum, aluminum chlorohydrate, aluminum sulphate, calcium oxide, calcium hydroxide, iron chloride, iron sulfate, polyacrylamide, sodium aluminate, sodium silicate, and combinations thereof.

Embodiment 9. The method of embodiment 7, wherein the flocculating agent comprises iron chloride.

Embodiment 10. The method of embodiment 9, further comprising slurrying the de-oiled algae bodies with water to between about 25% to about 50% by weight water and reducing the de-oiled algae bodies in size to an average about 15 microns to about 100 microns prior to pumping the de-oiled algae bodies.

Embodiment 11. The method of any of embodiments 2-10, further comprising: introducing an algae growth medium stream into an algae bioreactor; growing algae in the algae bioreactor; and withdrawing an algae stream from the algae bioreactor.

Embodiment 12. The method of embodiment 11, further comprising: introducing the algae stream into an extraction unit and extracting a bioproduct from algae to generate the de-oiled algae bodies.

Embodiment 13. A method comprising: providing a de-oiled algae body stream comprising de-oiled algae bodies; and pumping the de-oiled algae bodies into an underground storage location.

Embodiment 14. The method of embodiment 13, further comprising processing the de-oiled algae bodies by de-watering the de-oiled algae bodies prior to pumping the de-oiled algae bodies.

Embodiment 15. The method of any of embodiments 13-14, wherein the de-oiled algae bodies are slurried with water to between about 25% to about 50% by weight water prior to pumping the de-oiled algae bodies.

Embodiment 16. The method of any of embodiments 13-15, further comprising reduced the de-oiled algae bodies to between about 1 micron to about 100 microns in size prior to pumping the de-oiled algae bodies.

Embodiment 17. The method of any of embodiments 13-16, further comprising adding a flocculating agent to the de-oiled algae bodies prior to pumping the de-oiled algae bodies.

Embodiment 18. The method of embodiment 17, wherein the flocculating agent is selected from the group consisting of alum, aluminum chlorohydrate, aluminum sulphate, calcium oxide, calcium hydroxide, iron chloride, iron sulfate, polyacrylamide, sodium aluminate, sodium silicate, and combinations thereof.

Embodiment 19. A method comprising: providing a de-oiled algae body stream comprising de-oiled algae bodies; processing the de-oiled algae bodies by slurrying the de-oiled algae bodies with water to produce an algae body disposal stream; and pumping the de-oiled algae bodies into a salt cavern.

Embodiment 20. The method of embodiment 19, wherein the de-oiled algae bodies are slurried with water to between about 25% to about 50% by weight water, and further wherein the de-oiled algae bodies are reduced in size to an average of less than 15 microns, wherein a flocculating agent is added to the de-oiled algae bodies, and wherein the flocculating agent is selected from the group consisting of alum, aluminum chlorohydrate, aluminum sulphate, calcium oxide, calcium hydroxide, iron chloride, iron sulfate, polyacrylamide, sodium aluminate, sodium silicate, and combinations thereof.

While the disclosure has been described with respect to a number of embodiments and examples, those skilled in the art, having benefit of this disclosure, will appreciate that other embodiments can be devised which do not depart from the scope and spirit of the disclosure as disclosed herein. Although individual embodiments are discussed, the present disclosure covers all combinations of all those embodiments.

While compositions, methods, and processes are described herein in terms of “comprising,” “containing,” “having,” or “including” various components or steps, the compositions and methods can also “consist essentially of” or “consist of” the various components and steps. The phrases, unless otherwise specified, “consists essentially of” and “consisting essentially of” do not exclude the presence of other steps, elements, or materials, whether or not, specifically mentioned in this specification, so long as such steps, elements, or materials, do not affect the basic and novel characteristics of the disclosure, additionally, they do not exclude impurities and variances normally associated with the elements and materials used.

All numerical values within the detailed description and the claims herein modified by “about” or “approximately” with respect the indicated value are intended to take into account experimental error and variations that would be expected by a person having ordinary skill in the art.

For the sake of brevity, only certain ranges are explicitly disclosed herein. However, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited.

1. A method comprising:
providing a de-oiled algae body stream comprising de-oiled algae bodies; and
pumping the de-oiled algae bodies into a salt cavern.

2. The method of claim 1, further comprising processing the de-oiled algae bodies by de-watering the de-oiled algae bodies prior to pumping the de-oiled algae bodies.

3. The method of claim 1, further comprising slurrying the de-oiled algae bodies with water prior to pumping the de-oiled algae bodies.

4. The method of claim 3, wherein the de-oiled algae bodies are slurried with water to between about 25% to about 50% by weight water.

5. The method of claim 1, further comprising reducing the de-oiled algae bodies in size prior to pumping the de-oiled algae bodies.

6. The method of claim 5, wherein the de-oiled algae bodies are reduced in size to an average of less than about 15 microns prior to pumping the de-oiled algae bodies.

7. The method of claim 1, further comprising adding a flocculating agent to the de-oiled algae bodies prior to pumping the de-oiled algae bodies.

8. The method of claim 7, wherein the flocculating agent is selected from the group consisting of alum, aluminum chlorohydrate, aluminum sulphate, calcium oxide, calcium hydroxide, iron chloride, iron sulfate, polyacrylamide, sodium aluminate, sodium silicate, and combinations thereof.

9. The method of claim 7, wherein the flocculating agent comprises iron chloride.

10. The method of claim 9, further comprising slurrying the de-oiled algae bodies with water to between about 25% to about 50% by weight water and reducing the de-oiled algae bodies in size to an average about 15 microns to about 100 microns prior to pumping the de-oiled algae bodies.

11. The method of claim 1, further comprising:
introducing an algae growth medium stream into an algae bioreactor;
growing algae in the algae bioreactor; and
withdrawing an algae stream from the algae bioreactor.

12. The method of claim 11, further comprising:
introducing the algae stream into an extraction unit and extracting a bioproduct from algae to generate the de-oiled algae bodies.

13. A method comprising:
providing a de-oiled algae body stream comprising de-oiled algae bodies; and
pumping the de-oiled algae bodies into an underground storage location.

14. The method of claim 13, further comprising processing the de-oiled algae bodies by de-watering the de-oiled algae bodies prior to pumping the de-oiled algae bodies.

15. The method of claim 13, wherein the de-oiled algae bodies are slurried with water to between about 25% to about 50% by weight water prior to pumping the de-oiled algae bodies.

16. The method of claim 13, further comprising reduced the de-oiled algae bodies to between about 1 micron to about 100 microns in size prior to pumping the de-oiled algae bodies.

17. The method of claim 13, further comprising adding a flocculating agent to the de-oiled algae bodies prior to pumping the de-oiled algae bodies.

18. The method of claim 17, wherein the flocculating agent is selected from the group consisting of alum, aluminum chlorohydrate, aluminum sulphate, calcium oxide, calcium hydroxide, iron chloride, iron sulfate, polyacrylamide, sodium aluminate, sodium silicate, and combinations thereof.

19. A method comprising:
providing a de-oiled algae body stream comprising de-oiled algae bodies;
processing the de-oiled algae bodies by slurrying the de-oiled algae bodies with water to produce an algae body disposal stream; and
pumping the de-oiled algae bodies into a salt cavern.

20. The method of claim 19, wherein the de-oiled algae bodies are slurried with water to between about 25% to about 50% by weight water, and further wherein the de-oiled algae bodies are reduced in size to an average of less than 15 microns, wherein a flocculating agent is added to the de-oiled algae bodies, and wherein the flocculating agent is selected from the group consisting of alum, aluminum chlorohydrate, aluminum sulphate, calcium oxide, calcium hydroxide, iron chloride, iron sulfate, polyacrylamide, sodium aluminate, sodium silicate, and combinations thereof.
